(19) United States
(12) Patent Application Publication
(10) Pub. No.: US 2016/0007857 A1
(43) Pub. Date: Jan. 14, 2016

(54) SYSTEMS AND METHODS OF CREATING IN VIVO MEDICAL IMAGES OF TISSUE NEAR A CAVITY

(71) Applicant: University of Washington, Seattle, WA (US)

(72) Inventors: Ruikang K. Wang, Seattle, WA (US); Woo June Choi, Seattle, WA (US)

(21) Appl. No.: 14/796,925

(22) Filed: Jul. 10, 2015

Related U.S. Application Data
(60) Provisional application No. 62/023,723, filed on Jul. 11, 2014.

(51) Int. Cl. A61B 5/00; A61B 5/026

(52) U.S. Cl. CPC A61B 5/0073; A61B 5/0261; A61B 5/0066; A61B 5/6867

(57) ABSTRACT

Systems and methods of forming optical coherence tomography (OCT) images of tissue near a cavity of subject are disclosed herein. In one embodiment, a method of forming an image includes transmitting light pulses toward a region of interest near the cavity and receiving light backscattered from the region of interest using an imaging probe. The imaging probe includes a nosepiece configured to be at least partially received into the cavity. An image of the region of interest is formed using the backscattered light received from the region of interest.

### CROSS-REFERENCE TO RELATED APPLICATION(S)

This application claims the benefit of pending U.S. Provisional Application No. 62/023,723, filed Jul. 11, 2014. This application is also related to commonly owned International Application No. PCT/US2014/033297, filed Apr. 8, 2014. The foregoing applications are both incorporated herein by reference in their entireties.

### STATEMENT REGARDING FEDERALLY SPONSORED RESEARCH

This invention was made with government support under Grant No. R01DC010201, awarded by the National Institute on Deafness and Other Communication Disorders. The government has certain rights in the invention.

### TECHNICAL FIELD

The present application generally relates to medical imaging. In particular, several embodiments include systems and methods of constructing images of tissue surrounding and/or proximate a cavity of a subject.

### BACKGROUND

Diseases in cavities, such as the mouth and nose, are a prevalent and significant health care problem worldwide. For instance, it is estimated that over 2.5% of all new cancer cases in the United States occur in the oral cavity and pharynx. Moreover, for under-privileged groups in the developed or developing countries, cavity-related diseases have been one of the most important health burdens in terms of prevalence, severity and associated healthcare costs. In addition to low public awareness of these diseases, conventional visual examination methods used by physicians have hindered opportunities to manage the diseases at early stages because of subjective criteria of the examiners.

### BRIEF DESCRIPTION OF THE DRAWINGS

FIGS. 1A and 1B are block diagrams of an imaging system configured in accordance with an embodiment of the disclosed technology.

FIG. 2A is front isometric view of an imaging probe configured in accordance with an embodiment of the disclosed technology.

FIG. 2B is a cross sectional side schematic view of a portion of the imaging probe of FIG. 2A.

FIG. 3A is front isometric view of an imaging probe configured in accordance with another embodiment of the disclosed technology.

FIG. 3B is a cross sectional side schematic view of a portion of the imaging probe of FIG. 3A.

FIG. 4 is a flow diagram of a method of forming images configured in accordance with an embodiment of the disclosed technology.

FIG. 5 is a flow diagram of a method of forming images configured in accordance with an embodiment of the disclosed technology.

FIG. 6A shows a medical image formed in accordance with an embodiment of the disclosed technology.

FIGS. 6B-6D are medical images along an image slice of the medical image of FIG. 6A.

FIG. 6E is a medical image formed along the A-A′ line of FIG. 6A.

FIG. 6F is a medical image formed along the B-B′ line of FIG. 6A.

### DETAILED DESCRIPTION

The present disclosure relates generally to forming images of tissue surrounding and/or proximate a cavity of a subject. In one embodiment of the disclosed technology, for example, a plurality of light pulses are transmitted from a laser light source toward a region of interest. Backscattered light is received from the region of interest via an imaging probe having a nosepiece configured to be at least partially received into the cavity. The backscattered light is received at a detector optically coupled to the imaging probe, and first and second sets of image data are acquired using a portion of the backscattered light received at the detector. The first and second sets of image data are combined to form a plurality of blood flow image frames, and a three-dimensional image of the region of interest is constructed using the blood flow image frames. In some aspects, the nosepiece includes a first aperture at a proximal end portion of the nosepiece and a second aperture between the proximal and distal end portions of the nosepiece. In these aspects, transmitting the plurality of light pulses also includes deflecting the light pulses through the second aperture and toward the region of interest. In other aspects, the nosepiece is removably attachable to a distal end portion of the imaging probe.

In another embodiment of the disclosed technology, a method of determining a blood perfusion through a region of interest proximate an interior surface of an anatomical cavity of a subject includes transmitting laser light toward the region of interest and receiving light backscattered from the region of interest via an attachment on the imaging probe configured to be at least partially inserted into the anatomical cavity. Volumetric data is acquired from a portion of the received backscattered light, and a plurality of flow intensity image frames are acquired using the volumetric data. A mask is applied to the plurality of flow intensity image frames to form a plurality of masked image frames that are combined to construct a graphical representation of blood perfusion through the region of interest.

In yet another embodiment of the disclosed technology, a medical imaging system includes a light source configured to produce laser light and an interferometer optically coupled to the light source. The interferometer is further coupled to an imaging probe including a cavity measurement assembly removably attached to the imaging probe. The cavity measurement assembly is configured to be received in a cavity of the subject. The imaging probe and the cavity measurement assembly are configured to transmit laser light from the interferometer toward a region of interest near the cavity. The imaging probe and the cavity measurement assembly are also configured to convey light backscattered from the region of interest toward the interferometer. A detector optically coupled to the interferometer is configured to produce electrical signals corresponding to light signals received from the interferometer. A processor and memory are operatively coupled to the detector. The memory includes instructions executable by the processor to form a vascular image of the region of interest using electrical signals produced by the detector. In some aspects, the cavity measurement assembly includes an aperture between a proximal and distal end portions of the assembly, and a prism radially aligned with the aperture.

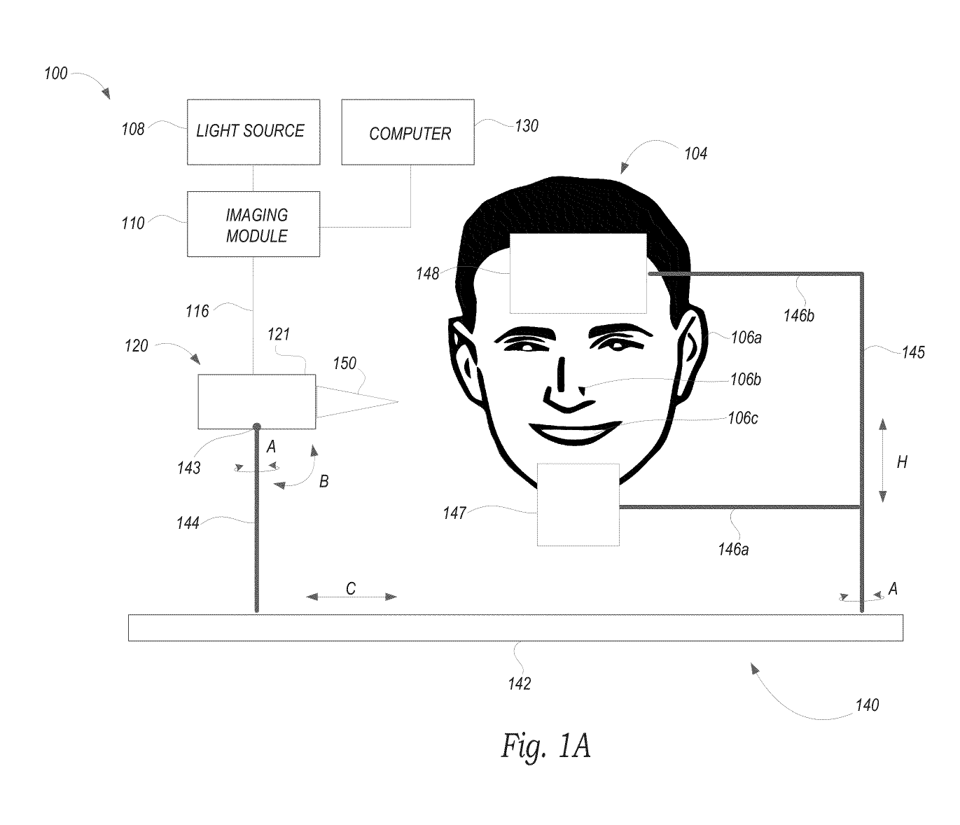

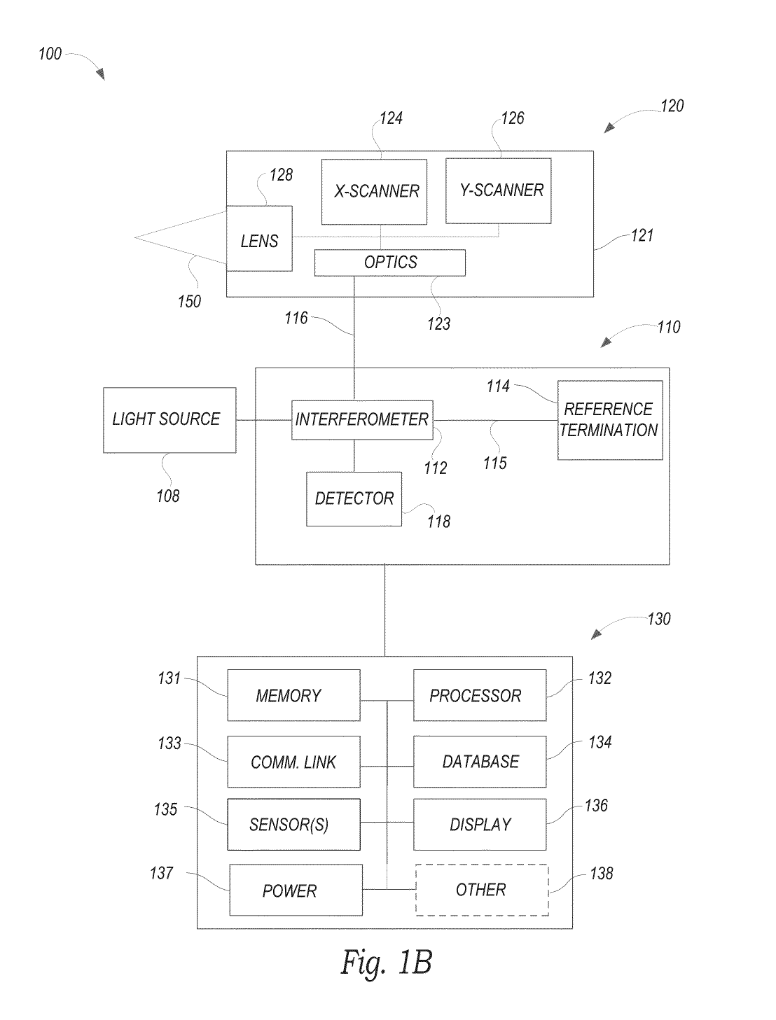

These and other aspects of the present disclosure are described in greater detail below. Certain details are set forth in the following description and in FIGS. 1-6F to provide a thorough understanding of various embodiments of the disclosure. Other details describing well-known systems and methods often associated with medical imaging and/or optical coherence tomography (“OCT” hereinafter), have not been set forth in the following disclosure to avoid unnecessarily obscuring the description of the various embodiments.

Many of the details, dimensions, angles and other features shown in the Figures are merely illustrative of particular embodiments of the disclosed technology. Accordingly, other embodiments can have other details, dimensions, angles and features without departing from the spirit or scope of the disclosure. In addition, those of ordinary skill in the art will appreciate that further embodiments of the invention can be practiced without several of the details described below.

In the Figures, identical reference numbers identify identical, or at least generally similar, elements. To facilitate the discussion of any particular element, the most significant digit or digits of any reference number refers to the Figure in which that element is first introduced. For example, element 110 is first introduced and discussed with reference to FIG. 1.

### Suitable Systems

FIG. 1A is a block diagram of an imaging system 100 configured in accordance with an embodiment of the disclosed technology. The system 100 includes an imaging module 110 coupled to a light source 108 and a computer or a processing subsystem 130. A sample arm 116 (e.g., a cable comprising one or more optical fibers) couples an imaging probe 120 to the imaging module 110. The probe 120 includes a nosepiece 150 attached to an end portion of a housing 121. The nosepiece 150 is configured to be received into a human subject's natural orifice and/or anatomical cavity (e.g., ear canal, nostril, mouth, vaginal cavity, rectal cavity). As explained in further detail below, the system 100 is configured to produce optical coherence tomography (OCT) images of tissue near a cavity using light backscattered from the tissue via the nosepiece 150. The backscattered light can be used to form OCT images that show a flow of blood through the tissue.

The system 100 further includes a scanning platform 140 configured to carry the probe 120 and to receive and hold a subject 104 to facilitate imaging, for example, of an ear 106a, a nostril 106b and/or a mouth 106c of the subject 104 during an imaging procedure. The scanning platform 140 includes a first support member 144 and a second support member 145 rotationally coupled to a base portion 142. The first support member 144 and second support member 145 are configured to rotate relative to the base portion 142 in the directions indicated by the arrows A. In some embodiments, the first support member 144 and second support member 145 can be configured to move laterally (e.g., along one or more rails (not shown)) relative to the base portion 142 in the direction indicated by the arrow C. A coupler 143 (e.g., a hinge, a pivot) attaches the probe 120 to the first support member 144 and is configured to allow the probe 120 to rotate in the directions indicated by the arrows A and B to facilitate placement of the probe 120 during imaging procedures. A first arm 146a and a second arm 146b extend from the second support member 145. A chin rest 147 carried by the first arm 146a and a forehead rest 148 carried by the second arm 146b are configured to receive and hold the chin and forehead, respectively, of a subject 104 during an imaging procedure. The first arm 146a and the second arm 146b are configured to be adjustable in the direction indicated by the arrow H to accommodate subjects with different-sized heads.

FIG. 1B is a block diagram of the system 100 showing certain components of the system 100 in more detail. As best seen in FIG. 1B, optics 123 optically couples the probe 120 to the imaging module 110. The optics 123 may include, for example, one or more lenses, collimators, splitters, prisms and/or optical filters. In some embodiments, the optics 123 can include an optical filter configured to attenuate noise and other artifacts caused by reflections along a cavity. For example, nose hair along a nostril surface and/or fluids along an interior surface of a mouth may cause reflections that can affect image quality. An x-scanner 124 and a y-scanner 126 (e.g., x-y galvanometric scanners) in the probe 120 are configured to perform scans of a region of interest in the subject. A lens 128 optically couples the optics 123, the x-scanner 124, and the y-scanner 126 to the nosepiece 150. The lens 128 is configured to focus and/or direct laser light received from the light source 108 via the imaging module 110 toward the region of interest. The lens 128 is further configured to direct backscattered light received from the region of interest toward the x-scanner 124 and/or the y-scanner 126. In some embodiments, the lens 128 includes a 5× telecentric lens. In one embodiment, the lens 128 may include, for example, an LSMO3 lens having a working distance of 25.1 mm and manufactured by Thorlabs Inc. In other embodiments, however, the lens 128 can include any lens suitable for OCT imaging.

The light source 108 includes a swept-source laser configured to output laser light. The light source 108 can be configured, for example, to sweep the laser wavelength across a broad spectral range near 1300 nm at a fixed repetition rate of 100 kHz. In some embodiments, the light source 108 includes a MEMS-tunable vertical cavity surface-emitting laser. In one embodiment, the light source 108 includes for example, a SL1310V1-10048 model laser manufactured by Thorlabs Inc. In other embodiments, however, the light source 108 may include any light source suitable for OCT imaging. The light source 108 is configured to emit an output beam (e.g., a 28 mW laser output beam) toward an interferometer 112 in the imaging module 110 optically coupled to the probe 120 via the sample arm 116. The interferometer 112 (e.g., a Mach-Zehnder interferometer and/or any suitable Michelson-type interferometer) is coupled to a reference 114 (e.g., a mirror) via a reference arm 115 (e.g., a cable, a conduit and/or one or more optical fibers). A detector 118 (e.g., a gain-balanced photo-detector) is optically coupled to the interferometer 112 via optics 119 (e.g., one or more lens, collimators, beam splitters, diffraction gratings, transmission gratings). The detector 118 is configured to produce one or more electrical signals that generally correspond to and/or are indicative of intensities of light signals received from the interferometer 112. In some embodiments, the light signals include an interference signal resulting from a combination in the interferometer 112 of light reflected from the reference 114 and backscattered light received from the region of interest via the probe 120. As described in further detail below, the processing subsystem 130 is configured to receive the electrical signals produced by the detector 118 and acquire one or more sets of image data to produce one or more medical images.

### Processing Subsystem

The following discussion provides a brief, general description of a suitable environment in which the technology may be implemented. Although not required, aspects of the technology are described in the general context of computer-executable instructions, such as routines executed by a general-purpose computer. Aspects of the technology can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network (e.g., a wireless communication network, a wired communication network, a cellular communication network, the Internet, a short-range radio network (e.g., via Bluetooth)). In a distributed computing environment, program modules may be located in both local and remote memory storage devices.

Computer-implemented instructions, data structures, screen displays, and other data under aspects of the technology may be stored or distributed on computer-readable storage media, including magnetically or optically readable computer disks, as microcode on semiconductor memory, nanotechnology memory, organic or optical memory, or other portable and/or non-transitory data storage media. In some embodiments, aspects of the technology may be distributed over the Internet or over other networks (e.g. a Bluetooth network) on a propagated signal on a propagation medium (e.g., an electromagnetic wave(s), a sound wave) over a period of time, or may be provided on any analog or digital network (packet switched, circuit switched, or other scheme).

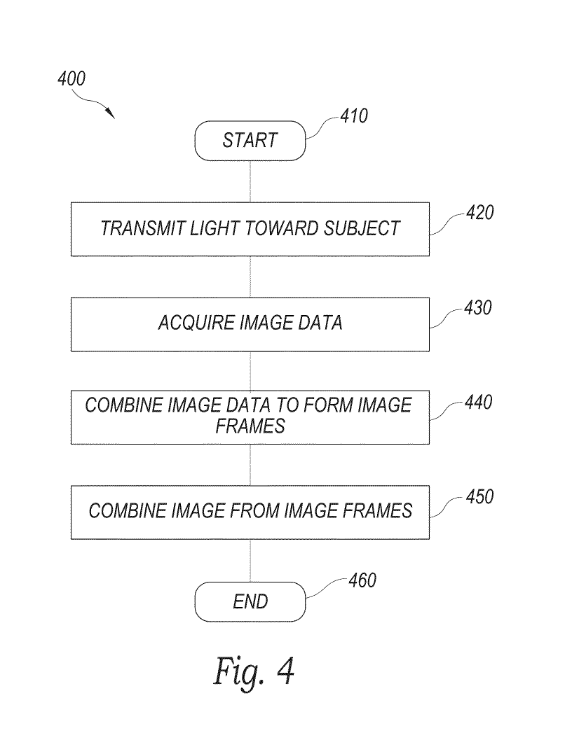

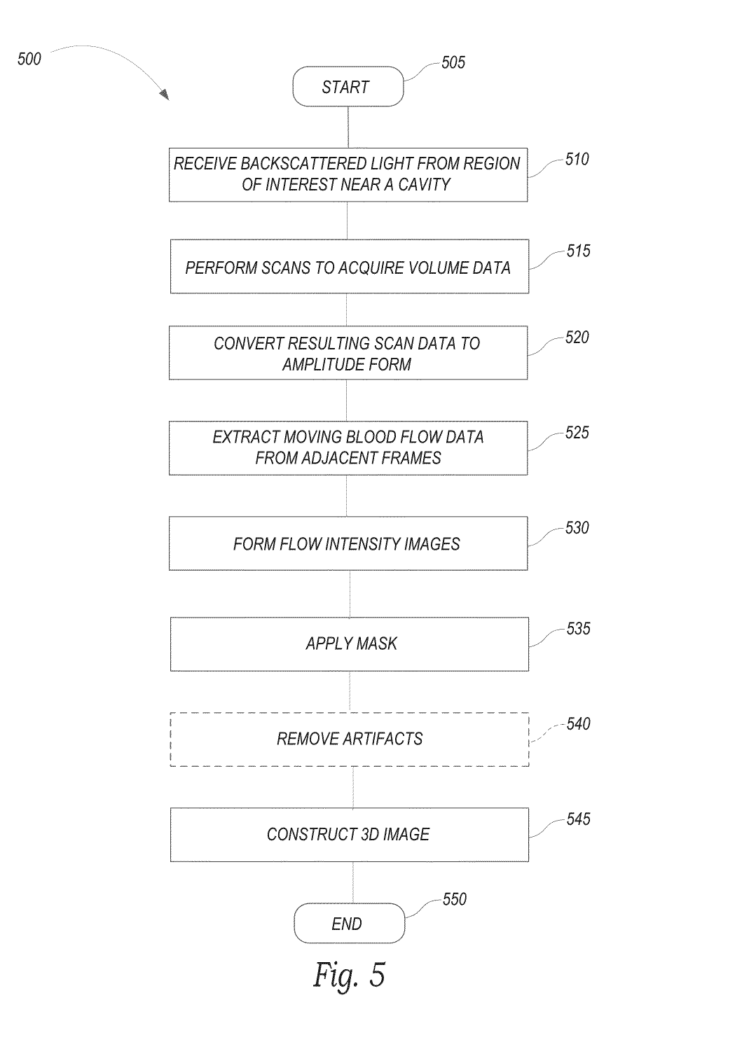

Referring still to FIG. 1B, the processing subsystem 130 includes several components including memory 131 (e.g., one or more computer readable storage modules, components, devices) and one or more processors 132. The memory 131 can be configured to store information (e.g., image data, subject information or profiles, environmental data, data collected from one or more sensors, media files) and/or executable instructions that can be executed by the one or more processors 132. As explained in further detail below in reference to FIGS. 4 and 5, the memory 131 can include, for example, instructions for forming, processing or otherwise constructing medical images of a region of interest using electrical signals produced by the detector 118 that are indicative of intensities of coherent backscattered light received from the region of interest. The medical images may include, for example, one or more two-dimensional images, three-dimensionals images and/or video clips comprising a graphical representation of blood perfusion and/or vascular architecture of the region of interest.

The processing subsystem 130 also includes communication components 133 (e.g., a wired communication link and/or a wireless communication link (e.g., Bluetooth, Wi-Fi, infrared and/or another wireless radio transmission network)) and a database 134 configured to store to data (e.g., image data acquired from the region of interest, equations, filters) used in the generation of medical images. One or more sensors 135 can provide additional data for use in image processing and/or construction. The one or more sensors 135 may include, for example, one or more ECG sensors, blood pressure monitors, galvanometers, accelerometers, thermometers, hygrometers, blood pressure sensors, altimeters, gyroscopes, magnetometers, proximity sensors, barometers and/or hall effect sensors. One or more displays 136 can provide video output and/or graphical representations of images formed by the system 100. A power supply 137 (e.g., a power cable connected to a building power system, one or more batteries and/or capacitors) can provide electrical power to components of the processing subsystem 130 and/or the system 100. In embodiments that include one or more batteries, the power supply 137 can be configured to recharge, for example, via a power cable, inductive charging, and/or another suitable recharging method. Furthermore, in some embodiments, the processing subsystem 130 may one or more additional components 138 (e.g., one or more microphones, cameras, Global Positioning System (GPS) sensors, Near Field Communication (NFC) sensors).

In some embodiments, the processing subsystem 130 may comprise one or more components that are partially or wholly incorporated into the imaging module 110 and/or the probe 120. In other embodiments, however, the processing subsystem 130 may include components that are remote from the imaging module 110 and/or the probe 120 and connected thereto by a communication network (e.g., the Internet and/or another network). In some embodiments, for example, at least a portion of the processing subsystem 130 may reside on a mobile device (e.g., a mobile phone, a tablet, a personal digital assistant) and/or a computer (e.g., a desktop computer, a laptop) communicatively coupled to the imaging module 110 and/or the probe 120.

### Imaging Probes

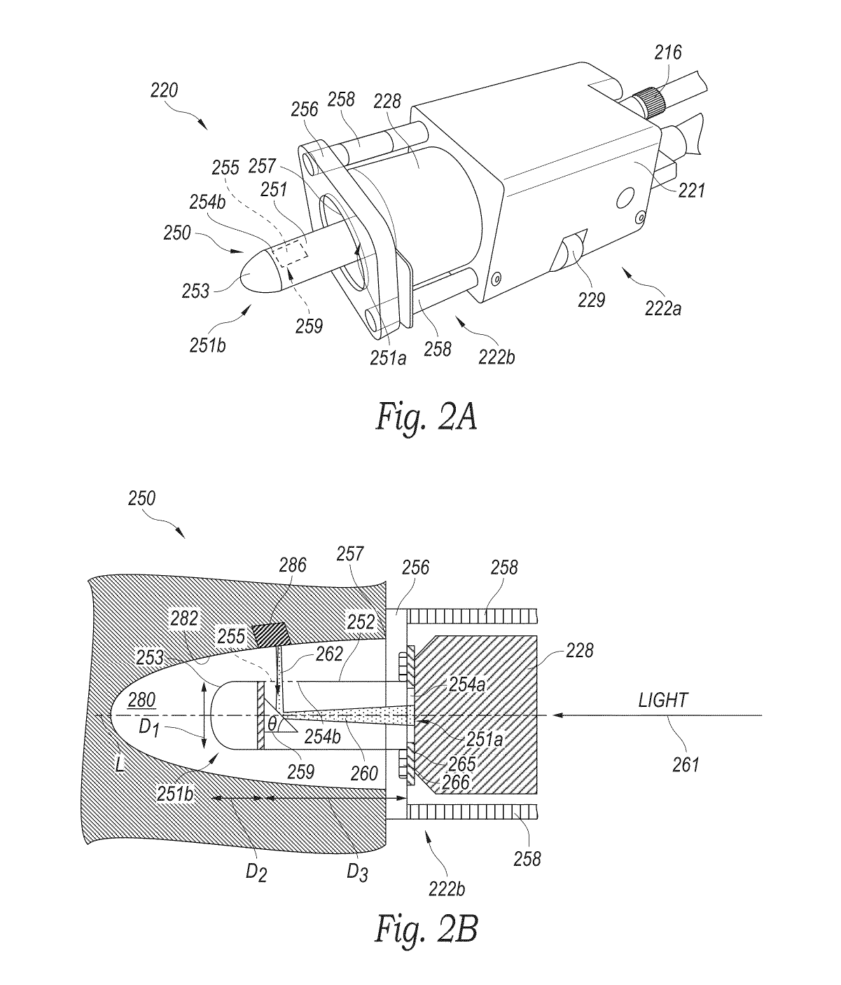

FIG. 2A is front isometric view of an imaging probe 220 (e.g., the probe 120 of FIGS. 1A and 1B) configured in accordance with an embodiment of the disclosed technology. FIG. 2B is a side schematic view of the probe 220 having an attachment, nosepiece or cavity measurement assembly 250 configured to be at least partially inserted into a cavity 280 of a subject. Referring to FIGS. 2A and 2B together, the probe 220 includes an enclosure or a housing 221 extending from a proximal end portion 222a to a distal end portion 222b. A cable 216 optically and/or electrically couples the probe 220 to a light source and/or an imaging module (e.g., the light source 108 and/or the imaging module 110 of FIGS. 1A and 1B). A lens 228 (e.g., the lens 128 of FIGS. 1A and 1B) is configured to transmit laser light from the cable 216 toward a region of interest 286 in the subject and directs backscattered light from the region of interest 286 toward the cable 216 and/or one or more x-y scanners (e.g., the x-scanner 124 and/or the y-scanner 126 of FIG. 1A). A control 229 (e.g., a dial, a knob and/or a switch) can be configured to control an intensity of laser light transmitted via the probe 220 toward the region of interest 286. A plurality of spacer bars 258 extend through the housing 221 toward a front plate 256 disposed at the distal end portion 222b of the housing 221. The spacer bars 258 are positioned to maintain a constant distance between the lens 228 and the region of interest 286. A plate aperture 257 formed in the front plate 256 allows the assembly 250 to pass therethrough.

The assembly 250 includes a proximal end portion 251a, a distal end portion 251b, an intermediate portion 252 and a longitudinal axis L (FIG. 2B) extending therethrough. An end cap 253 at the distal end portion 251b prevents liquids or other contaminants from entering the assembly 250. The proximal end portion 251a of the assembly 250 is attached to the distal end portion 222b of the housing 221 via the plate aperture 257. A first aperture 254a (FIG. 2B) at the proximal end portion 251 and a second aperture 254b in the intermediate portion 252 allow laser light from the lens 228 to pass through the assembly 250 toward the region of interest 286 in the subject. As discussed in more detail below in reference to FIG. 2B, a deflector 259 (e.g., a prism) disposed in the intermediate portion 252 is configured to deflect laser light from the lens 228 through a transparent, sterilized sheath 255 toward the region of interest 286 and deflect backscattered light from the region of interest 286 toward the lens 228.

In some embodiments, the assembly 250 is removably attached to the housing 221 with a screw-on mechanism, an interference lock mechanism, one or more magnets, one or more tabbed inserts, an adhesive, etc. In other embodiments, however, the assembly 250 is fixedly attached or secured to the housing 221. In the illustrated embodiment, the assembly 250 comprises a translucent, sterilizable material (e.g., plastic). In other embodiments, however, the assembly 250 can comprise any suitable material (e.g., plastic, metal, glass). In some embodiments, the assembly 250 is configured to be disposable. Moreover, in the illustrated embodiment of FIGS. 2A and 2B, the intermediate portion 252 has a generally cylindrical shape that is configured to be received into the cavity 280. In other embodiments, the intermediate portion 252 is configured to have a size and shape that is insertable and/or receivable into a portion of any anatomical cavity (e.g., one or more nostrils, mouths, ears, vaginal cavities, a rectal cavities and/or urethras). In certain embodiments, the intermediate portion 252 can be configured to be positioned between adjacent digits (e.g., fingers, toes) of a subject's anatomy. In some other embodiments, however, the intermediate portion 252 can have a different suitable shape.

Referring now only to FIG. 2B, the proximal end portion 251a of the assembly 250 is disposed against a first annular ring 265 at the distal end portion 222b of the housing 221. A second annular ring 266 (e.g., a rubber ring) receives the proximal end portion 251a of the assembly 250 and is configured to restrict movement of the intermediate portion 252 when the assembly 250 is attached to the housing 221. The intermediate portion 252 has a height or diameter D₁ (e.g., between about 5 mm and about 25 mm, between about 7 mm and about 15 mm, or about 10 mm). The end cap 253 has a length D₂ (e.g., between about 3 mm and about 15 mm, between about 5 mm and about 10 mm, or about 7 mm) and the intermediate portion 252 has a length D₃ (e.g., between about 10 mm and about 40 mm, between about 15 mm and about 30 mm, or about 20 mm).

The deflector 259 has a right-triangular cross sectional shape with a complementary angle θ (e.g., between about 30 degrees and about 60 degrees, or about 45 degrees) such that incident light is deflected by the deflector 259 at an angle relative to the longitudinal axis L that is generally normal to an angle at which the deflector 259 receives the light. The deflector 259 is mounted on or otherwise attached to a member 266 disposed in the intermediate portion 252.

The member 266 is configured to maintain an alignment of the deflector 259 with laser light transmitted through the assembly 250 toward the region of interest 286 and light backscattered from the region of interest 286.

In operation, at least a portion of the assembly 250 is inserted into the cavity 280 (e.g., a nostril, mouth, ear) of the subject such that the second aperture 254b and the deflector 259 are substantially aligned in a radial direction (i.e., orthogonal to the longitudinal axis L) with the region of interest 286 on and/or proximate an interior surface 282 of the cavity 280. The lens 228 receives laser light 261 from a light source and/or an imaging module (e.g., the light source 108 and/or the imaging module 110 of FIGS. 1A and 1B) and directs focused laser light 260 through the intermediate portion 252 toward the deflector 259 and substantially aligned with the longitudinal axis L. The deflector 259 deflects the focused laser light 260 toward the region of interest 286 and receives backscattered light 262 from the region of interest 286. The deflector 259 deflects the backscattered light 262 toward the lens 228 and other components in the housing 221 (e.g., the x-scanner 124 and/or the y-scanner 126 of FIGS. 1A and 1B). As described in more detail below in reference to FIGS. 4 and 5, the backscattered light 262 can be used to construct one or more images, such as, for example, a blood flow intensity image, a microvasculature image, an optical microangiograph (OMAG) image. Embodiments of the present disclosure are expected to provide at least the advantages of forming images of a tissue near a cavity showing blood flow and/or microvasculature of the tissue having a higher resolution and/or lower cost than other approaches (e.g., endoscopes, ultrasound devices, OCT probes using catheters and/or other medical devices).

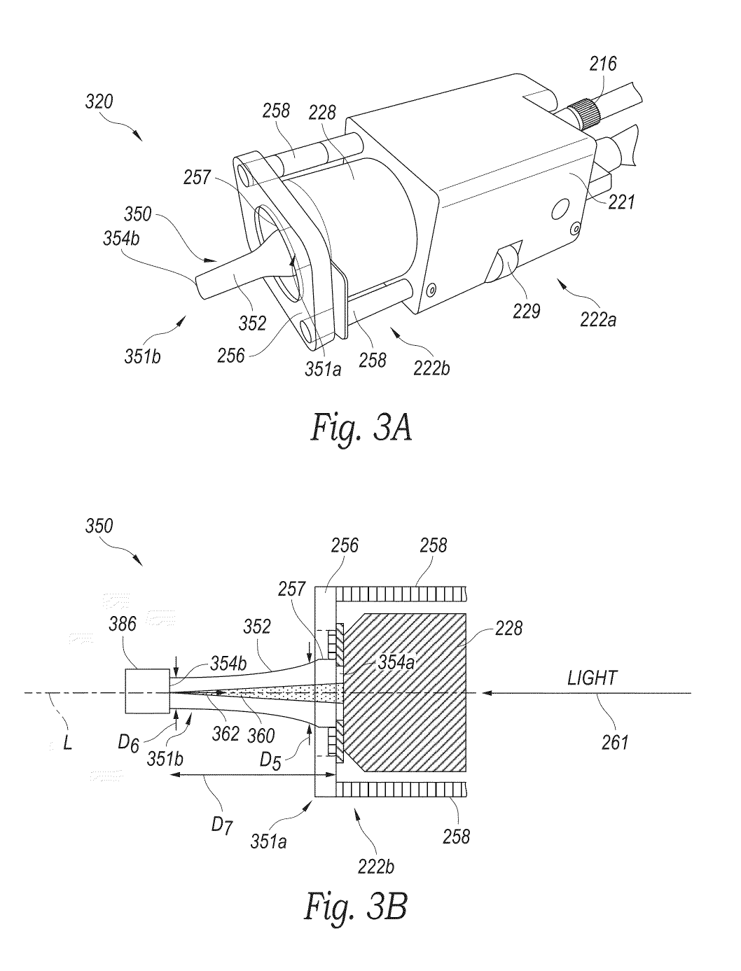

FIG. 3A is front isometric view of an imaging probe 320 configured in accordance with another embodiment of the disclosed technology. FIG. 3B is a side schematic view of an attachment, nosepiece or cavity measurement assembly 350 removably attached to the imaging probe of 320 shown in FIG. 3A. Referring to FIGS. 3A and 3B together, the assembly 350 includes a proximal end portion 351a, a distal end portion 351b, an intermediate portion 352 and a longitudinal axis L (FIG. 3B) extending therethrough. The proximal end portion 351a of the assembly 350 is attached to the distal end portion 222b of the housing 221 via the plate aperture 257. A first aperture 354a (FIG. 3B) at the proximal end portion 351 and a second aperture 354b in the intermediate portion 352 allow laser light from the lens 228 to pass through the assembly 350 toward a region of interest in a subject. In the illustrated embodiment, the assembly 350 comprises an opaque, sterilizable material (e.g., plastic). In other embodiments, however, the assembly 350 can comprise any suitable material (e.g., plastic, metal, glass). In some embodiments, the assembly 350 is configured to be disposable.

Referring now only to FIG. 3B, the assembly 350 is configured to be received into a portion of an anatomical cavity (e.g., one or more nostrils, mouth, ear(s), vaginal cavity, rectal cavity, urethra). The intermediate portion 352 has a shape (e.g., an aural speculum shape) that tapers from a first diameter D₅ (e.g., about 10 mm) toward a second diameter D₆ (e.g., about 5 mm), and a length D₇ (e.g., between about 10 mm and about 30 mm, between about 15 mm and about 25 mm or about 23 mm).

In operation, at least a portion of the assembly 350 is inserted into a cavity (not shown) such that the longitudinal axis L is substantially aligned with a portion of a region of interest 386 along and/or proximate an interior surface of the cavity. The lens 228 receives laser light 261 from a light source and/or an imaging module (e.g., the light source 108 and/or the imaging module 110 of FIGS. 1A and 1B) and directs focused laser light 360 toward the region of interest 386. Light 362 backscatters from the region of interest 386 toward the lens 228. An imaging module and/or processing subsystem (e.g., the imaging module 110 and/or the processing subsystem 130 of FIGS. 1A and 1B) coupled to the probe 220 receive the light 362 and form one or more medical images.

### Suitable Methods

FIG. 4 is a flow diagram showing a process 400 configured to form medical images in accordance with an embodiment of the disclosed technology. In some embodiments, the process 400 can comprise instructions stored, for example, on the memory 131 of the system 100 (FIG. 1B) that are executable by the one or more processors 132 (FIG. 1B). In some embodiments, portions of the process 400 are performed by one or more hardware components (e.g., the light source and/or the imaging module 110 of FIGS. 1A and 1B). In some embodiments, portions of the process 400 are performed by a device external to the system 100 of FIGS. 1A and 1B.

The process 400 begins at block 410. At block 420, the process 400 transmits light from a light source to optically coupled to an imaging probe (e.g., the light source 108 and the imaging probe 120 of FIGS. 1A and 1B). The imaging probe can include a nosepiece (e.g., the cavity measurement assemblies 250 and/or 350 of FIGS. 2A-3B) configured to be received into a subject's cavity (e.g., an ear, a nostril, a mouth, a vaginal cavity, a rectal cavity). The nosepiece directs the light toward a region of interest proximate an interior surface of the cavity.

At block 430, the process 400 acquires image data from the region of interest. As described in more detail in reference to FIG. 5, the process 400 can be configured to perform a plurality of image scans using backscattered light received from the cavity. In some embodiments, the process 400 can be configured to acquire A-line scans in one direction relative to the region of interest and B-frame scans in another direction relative to the region of interest. In certain embodiments, for example, the process 400 can perform a plurality of first scans in a first direction (e.g., an x-direction relative to the region of interest) and a plurality of second scans in a second, orthogonal direction (e.g., a y-direction relative to the region of interest).

At block 440, the process 400 combines the data acquired at block 430 to form a 3D dataset of the region of interest comprising a plurality (e.g., 256, 512, 1024) of optical microangiograph (OMAG) image frames. The process 400 also creates a correlation mapping OCT (cmOCT) image frame for each of the plurality of OMAG image frames.

At block 450, the process 400 multiplies each of the OMAG image frames with a corresponding cmOCT image frame to obtain a plurality of masked image frames that are combined to form a final image that includes, for example, a three-dimensional image showing blood perfusion through the region of interest. At block 460, the process ends.

FIG. 5 is a flow diagram of a process 500 configured to form medical images in accordance with another embodiment of the disclosed technology. In some embodiments, the process 500 can comprise instructions stored, for example, on the memory 131 of the system 100 (FIG. 1B) and executable by the one or more processors 132 (FIG. 1B). In some embodiments, portions of the process 500 are performed by one or more hardware components (e.g., the light source and/or the imaging module 110 of FIGS. 1A and 1B). In some embodiments, portions of the process 500 are performed by a device external to the system 100 of FIGS. 1A and 1B.

The process 500 begins at block 505. At block 510, backscattered light is received from a region of interest at a probe configured to be inserted into a cavity of a subject. At block 515, the process 500 performs a fast B-scan in an X-direction relative to the region of interest to obtain a B-frame containing a plurality (e.g., 256) of A-lines at an imaging rate (e.g., 100 frames/s (FPS), 200 FPS, 400 FPS). The process 500 further performs a slow C-scan in the Y-direction to obtain a plurality (e.g., 1024, 2048, 4096, 8192) of B-frames with a predetermined number (e.g., 2, 4, 8, 12) repetitions at the each location. Once the scans are completed, the process 500 produces volume data including an OCT data cube having a plurality of voxels corresponding to the region of interest.

At block 520, the process 500 converts the OCT data cube to amplitude form using, for example, a fast fourier transform (FFT). Continuing at block 525, the process 500 extracts moving blood flow data from adjacent B-frames in the amplitude data set to compensate, for example, for axial displacement induced by tissue bulk motion.

At block 530, the process 500 averages B-frames obtained at the same location to form a blood flow intensity image (i.e., an intensity-based optical microangiograph or IB-OMAG image) at each location. The process 500 repeats this calculation for each location of the plurality of location at which B-frames were obtained during the C-scan at block 520.

At block 535, the process 500 calculates a mask for each of the IB-OMAG images formed at block 530 to remove static artifacts. The process 500 calculates a correlation mapping OCT (cmOCT) (and/or another cross-correlation) between adjacent B-frames to provide a map of blood flow through the region of interest. The process 500 applies the mask by multiplying each of the IB-OMAG images with corresponding cmOCT images to obtain a plurality of masked OMAG (mOMAG) images.

At block 540, the process 500 may include removing artifacts from the images using, for example, one or more filters (e.g., a low-pass filter, a high pass filter, an optical filter). In some embodiments, for example, the process 500 may be configured to remove artifacts caused by respiration and/or pulsation movement of the subject. In other embodiments, the process 500 may be configured to filter out artifacts in an image caused by, for example, reflections of structures (e.g., hair) or fluids along a surface of the cavity proximate the region of interest. The artifact removal process at block 540 is an optional step that may not be included in some embodiments of the process 500.

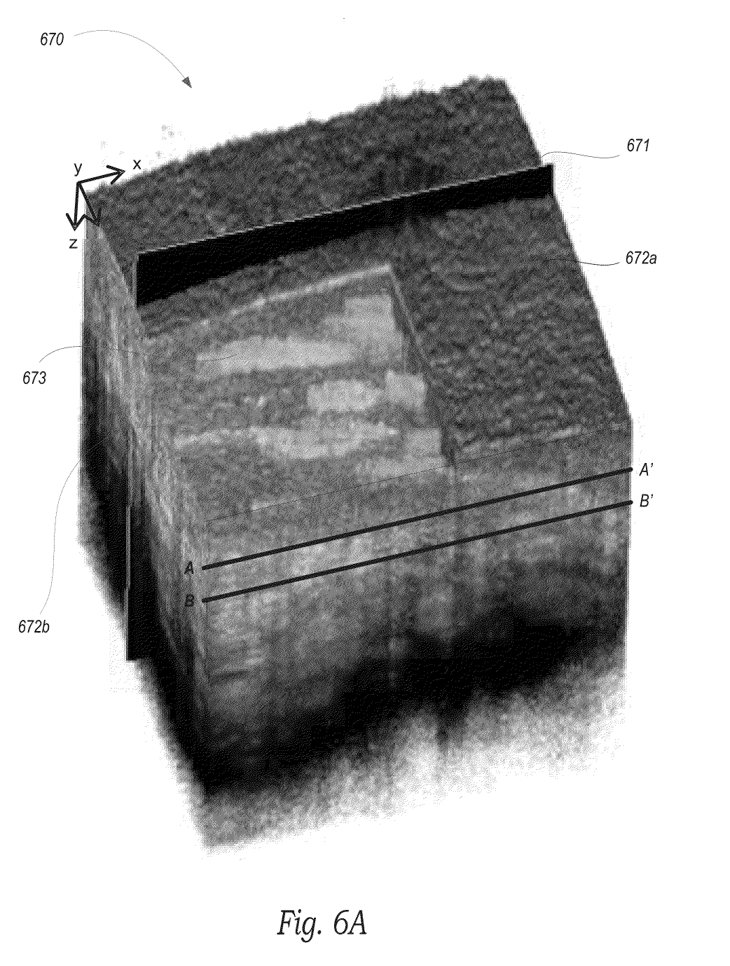

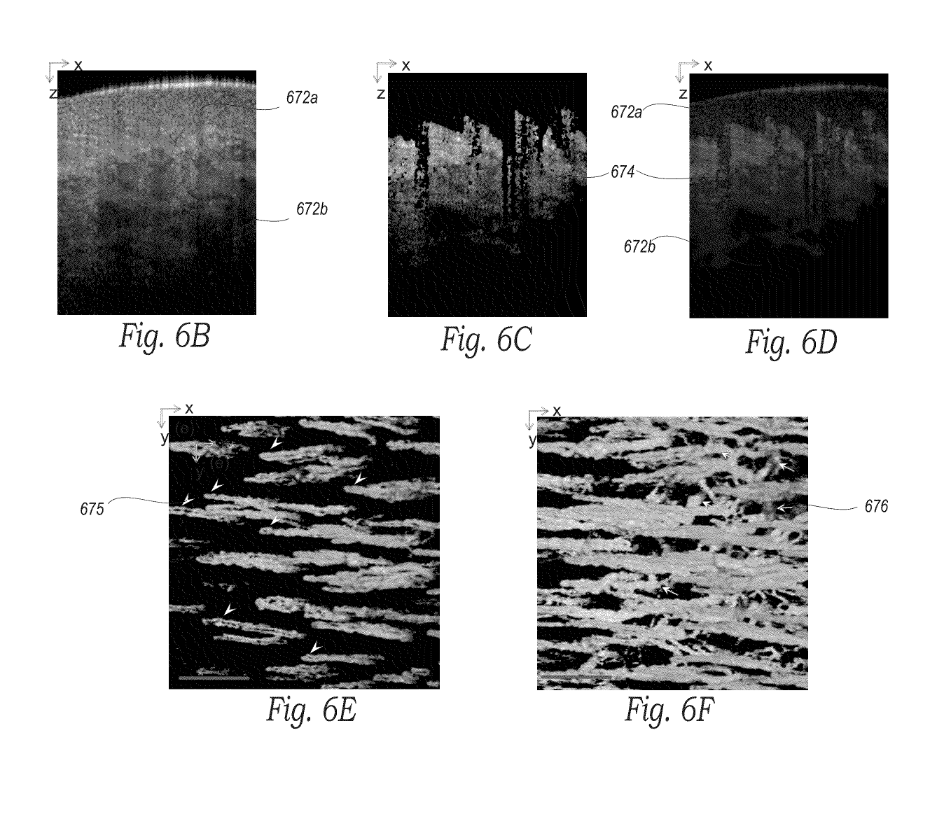

At block 545, the process 500 constructs one or more images using the mOMAG dataset. The images can include, for example, two-dimensional images, three-dimensional images and/or video images. Additional details regarding the process 500 and similar embodiments may be found, for example, in International Application No. PCT/US2014/033297, which has already been incorporated by reference above. Examples of images produced by the process 500 are shown in FIGS. 6A-6F, which are discussed below.

### Example

FIG. 6A shows a medical image 670 constructed in accordance with an embodiment of the disclosed technology. The image 670 includes a slice 671 in the X-Z plane along which image frames are acquired. The medical image 670 is a structural OCT image acquired from an interior surface of a subject's mouth and includes an epithelial layer 672a and an underlying lamina propria 672b. Overlay 673 includes OMAG image data showing a level of blood flow or perfusion through the region of interest. FIGS. 6B-6D are image frames formed along the slice 671 of the medical image 670. In particular, FIG. 6B is a structural OCT image frame and FIG. 6C is a OMAG image frame. FIG. 6D is formed by overlaying FIG. 6C onto FIG. 6B that shows locations of the blood vessels in the lamina propria 672b, making a clear demarcation from the avascular epithelial layer 672a.

FIG. 6E shows an image frame formed along the A-A′ of FIG. 6A at a first depth (e.g., approximately 250 microns). FIG. 6F shows an image frame formed along the B-B′ of FIG. 6A at a second, greater depth (approximately 410 microns). FIG. 6E shows hairpin-like capillary loops (indicated by arrow heads 675) that are visible near the junction of the epithelial layer 672a and the lamina propria 672b (FIGS. 6A and 6D) where they are arranged in parallel to the labial tissue surface. FIG. 6F shows capillaries emerging from wider planar arterioles (indicated by arrows 676). As those of ordinary skill in the art will appreciate, conventional 2D angiography techniques (e.g., capillaroscopy and sidestream darkfield microscopy) are not capable of resolving microvasculatures shown in FIG. 6F.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. Moreover, in some embodiments, the technology can be used to form images of tissue surrounding or near an anatomical cavity (e.g., a natural orifice such as one or more ears, nostrils, mouths, vaginal cavities, rectal cavities, urethras) of a subject (e.g., one or more humans or animals). As those of ordinary skill in the art will appreciate, an anatomical cavity as described herein does not comprise, for example, blood vessels (e.g., arteries, veins, fistulas) and/or regions in internal organs(e.g., a liver, kidney, heart) of a subject's body. In other embodiments, however, the technology may be used to form images of any portion of a subject's anatomy. The various embodiments described herein may also be combined to provide further embodiments.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Where the context permits, singular or plural terms may also include the plural or singular term, respectively. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A method of operating a medical imaging system to construct an optical coherence tomography (OCT) image of a region of interest proximate an interior surface of an anatomical cavity of a subject, the method comprising:
transmitting a plurality of light pulses from a laser light source toward the region of interest via a nosepiece attached to an imaging probe, wherein the nosepiece is configured to be at least partially received into the anatomical cavity;
receiving backscattered light from the region of interest at a detector optically coupled to the imaging probe;
acquiring first and second sets of image data using a portion of the backscattered light received at the detector;
combining the first and second sets of image data to form a plurality of blood flow image frames; and
constructing a three-dimensional image of the region of interest using the medical imaging system, wherein the three-dimensional image includes the plurality of blood flow image frames.

2. The method of claim 1 wherein transmitting the plurality of light pulses includes changing a path of the plurality of light pulses in the nosepiece by an angle relative to a longitudinal axis of the nosepiece.

3. The method of claim 1 wherein the nosepiece includes a proximal end portion and a distal end portion, and wherein the nosepiece further includes a first aperture at the proximal end portion, a second aperture between the proximal and distal end portions and a deflector radially aligned with the second aperture, and further wherein:
transmitting the plurality of light pulses includes deflecting the plurality of the light pulses via the deflector through the second aperture and toward the region of interest.

4. The method of claim 1 wherein the nosepiece is removably attachable to a distal end portion of the imaging probe.

5. The method of claim 1, further comprising receiving the subject's head at a scanning platform, wherein the scanning platform is configured to carry the imaging probe and further configured to hold the subject's head substantially still.

6. The method of claim 1 wherein acquiring the first set of image data includes performing a first number of scans along a first axis in the region of interest, and wherein acquiring the second set of image data includes performing a second number of scans along a second axis in the region of interest orthogonal to the first axis.

7. The method of claim 6 wherein acquiring the first set of image data includes acquiring the first number of A-line scans in the first direction and wherein acquiring the second set of image data includes acquiring the second number of B-frames in the second direction.

8. The method of claim 6 wherein:
acquiring the first set of image data includes performing a single scan at each of the first number of scanning positions along the first axis in the region of interest; and
acquiring second set of image data includes performing a predetermined number of scans at each of the first number of scanning positions along the second axis in the region of interest to form the predetermined number of B-frames at each scanning position, and wherein the predetermined number is greater than one.

9. The method of claim 8 wherein the individual blood flow images are formed by averaging B-frames in the second set of image data acquired at the same scanning position along the second axis in the region of interest.

10. The method of claim 1 wherein combining the first and second sets of image data further includes:
producing a combined set of image data from the first and second sets of image data; and
forming a plurality of correlation image frames by applying a correlation map to the combined set of image data, wherein individual correlation image frames directly correspond to one of the plurality of blood flow image frames.

11. The method of claim 10 wherein constructing the three-dimensional image includes arithmetically combining each correlation image frame with the corresponding blood flow image frame.

12. A method of determining blood perfusion through a region of interest proximate an interior surface of an anatomical cavity of a human subject, the method comprising:
transmitting laser light from a light source toward the region of interest;
receiving light backscattered from the region of interest via an attachment removably coupled to an imaging probe, wherein the attachment is configured to be at least partially inserted into the anatomical cavity;
acquiring volumetric data from a portion of the received backscattered light;
forming a plurality of flow intensity image frames using the volumetric data;
applying a mask to the plurality of flow intensity image frames to form a plurality of masked image frames; and
constructing a graphical representation of blood perfusion through the region of interest by combining the plurality of masked image frames.

13. The method of claim 12 wherein the attachment includes a proximal end portion, a distal end portion and a longitudinal axis extending therebetween, wherein the attachment further includes a first aperture at the proximal end portion, a second aperture between the proximal and distal end portions and a prism that axially overlaps the second aperture relative to the longitudinal axis of the attachment, and wherein receiving the backscattered light includes deflecting the backscattered light toward a lens in the imaging probe disposed proximate the proximal end portion of the attachment.

14. The method of claim 12 wherein acquiring the volumetric data includes:
acquiring a first number of A-lines along a first axis in the region of interest;
acquiring a plurality of B-frames at each of a second number of positions along a second axis in the region of interest, wherein the second axis is orthogonal to the first axis; and
averaging the plurality of B-frames acquired at each of the second number of positions to obtain the second number of averaged B-frames.

15. The method of claim 12 wherein applying a mask to the plurality of flow intensity images includes:
calculating an individual correlation image frame for each of the plurality of flow intensity images; and
arithmetically combining each correlation image frame with the corresponding flow intensity image frame.

16. The method of claim 12, further comprising applying a filter to the flow intensity image frames, wherein the filter is configured to reduce reflection artifacts caused by fluids on an interior surface of the cavity.

17. A medical imaging system configured to produce vascular images of a subject, the system comprising:
a light source configured to produce laser light;
an interferometer optically coupled to the light source, wherein the interferometer includes a first arm having a mirror, and a second arm, and wherein the interferometer is configured to split the laser light from the light source between the first arm and the second arm;
an imaging probe having a proximal end and a distal end, wherein the proximal end of the imaging probe is optically coupled to the second arm of the interferometer;
a cavity measurement assembly removably attached to the distal end of the imaging probe, wherein the cavity measurement assembly is configured to be received into a cavity of the subject,
wherein the imaging probe and the cavity measurement assembly are configured to transmit laser light from the second arm of the interferometer toward a region of interest proximate the cavity of the subject, and
wherein the imaging probe and the cavity measurement assembly are further configured to convey light backscattered from the region of interest toward the second arm of the interferometer;
a detector optically coupled to the interferometer, wherein the detector is configured to produce electrical signals that correspond to light signals received from the interferometer; and
a processor and memory operatively coupled to the detector, wherein the memory includes instructions executable by the processor to form a vascular image of the region of interest using the electrical signals produced by the detector.

18. The system of claim 17 wherein the cavity measurement assembly includes a proximal end portion, a distal end portion and longitudinal axis extending therebetween, wherein the cavity measurement assembly further includes an aperture between the proximal and distal end portions and a prism radially aligned with the aperture.

19. The system of claim 18, further comprising a sterile sheath disposed on the cavity measurement assembly, wherein the sterile sheath substantially covers the aperture.

20. The system of claim 17 wherein the cavity measurement assembly includes a proximal end portion, a distal end portion, and intermediate portion and longitudinal axis extending therebetween, wherein the diameter of intermediate portion tapers from a first diameter near the proximal end portion toward a second, lesser diameter near the distal end portion.
